(19) United States
(12) Patent Application Publication
(10) Pub. No.: US 2025/0191496 A1
(43) Pub. Date: Jun. 12, 2025

(54) TRAINING SYSTEM FOR SIMULATING COCHLEAR IMPLANT PROCEDURES

(71) Applicant: CARNEGIE MELLON UNIVERSITY, Pittsburgh, PA (US)

(72) Inventors: Maysamreza Chamanzar, Pittsburgh, PA (US); Jay Reddy, Pittsburgh, PA (US)

(73) Assignee: CARNEGIE MELLON UNIVERSITY, Pittsburgh, PA (US)

(21) Appl. No.: 18/844,243

(22) PCT Filed: Mar. 28, 2023
(86) PCT No.: PCT/US23/16522

§ 371 Date: Sep. 5, 2024

Related U.S. Application Data
(60) Provisional application No. 63/324,174, filed on Mar. 28, 2022.
(60) Provisional application No. 63/324,839, filed on Mar. 29, 2022.
(60) Provisional application No. 63/324,871, filed on Mar. 29, 2022.
(60) Provisional application No. 63/325,305, filed on Mar. 30, 2022.

(51) Int. Cl. G09B 23/28; A61N 1/36

(52) U.S. Cl. CPC G09B 23/285; A61N 1/36038

(57) ABSTRACT

Disclosed herein is a cochlear implant surgery simulator and training system. The system comprises a 3D model of a scala tympani upon which a surgeon performs practice insertions an the electrode array of a cochlear implant, wherein the electrode array is instrumented with one or more thin-film sensors disposed along a length of the electrode array, enabling real-time collection of force and position data. The system further comprises a feedback system for analyzing data collected from the instrumented electrode array and deriving scoring metrics regarding the surgeon's insertion technique.

### RELATED APPLICATIONS

This application claims the benefit of U.S. Provisional Patent Application Nos. 63/324,174, filed on Mar. 28, 2022, 63/324,839, filed Mar. 29, 2022, 63/324,871, filed Mar. 29, 2022 and 63/325,305, filed Mar. 30, 2022, the contents of which are incorporated herein in their entireties.

### BACKGROUND

When hearing loss progresses beyond the point where hearing aids are effective, acoustic hearing can be augmented with electronic hearing via a cochlear implant. These life-changing devices supplement acoustic hearing with electronic hearing by directly stimulating the auditory nerve.

FIG. 1 shows an in situ cochlear implant. As can be seen, the cochlea is a spiral-shaped structure through which an electrode array portion of the implant must be placed. The electrode array is threaded into the scala tympani chamber of the cochlea via a small hole drilled in the base of the cochlea. The electrode array consists of a plurality of electrodes, typically composed of platinum, embedded in a substrate, typically silicone. Although it varies from manufacturer to manufacturer, the electrode array may be approximately 0.6 mm in diameter and 2-3 cm in length. The electrode array is flexible to conform to the anatomy of the cochlea during insertion.

The electrode arrays are difficult to insert consistently. During insertion, the electrode tip can apply excessive forces to the inner surface of the scala tympani, destroying the fragile hair cells. Other adverse events include tip foldover or piercing the basilar membrane, resulting in scalar translocation. These surgical events can reduce the performance of the implant, as well as causing trauma and thereby destroying or diminishing any remaining acoustic hearing capability of the patient. Additionally, the precise placement of the cochlear implant in the scala tympani contributes to the outcome. The lack of tools to precisely control the final implant position causes outcome variability, which, along with residual hearing loss, is a major barrier to adoption amongst the current eligible population.

Measurable features collected during implantation can predict outcomes of the surgery. Such features include the cochlear implant placement, insertion force and structural damage. Currently, these features are sensed qualitatively by the surgeon and the surgeon can adjust the insertion, based on the sensed features. Expert surgeons have optimized the insertion technique to reduce trauma and preserve hearing based on subtle changes as the electrode array is implanted. Currently however, the surgeon's feedback of the insertion force is limited to the resistance they perceive as they manually thread the electrode array into the cochlea, which is limited to the sensitivity of human perception and is highly dependent on the surgeon's experience and dexterity.

Force-measurement systems in robotic platforms have been used to monitor external insertion force during surgery, but only measure the cumulative force and are unable to localize causes of increased insertion force. Additionally, while prior work has attempted to utilize MEMS technology to replace the cochlear implant electrode array, sensing capabilities using dedicated sensors integrated in to the electrode array are unknown in the art. There have also been attempts to enhance the surgeon's ability to actuate the electrode array precisely, either using robotically guided insertion, magnetic guidance systems, or built-in actuators. However, these techniques do not incorporate in situ feedback from the cochlear implant electrode array.

### SUMMARY

Described herein as a first aspect of the invention is a novel design of an instrumented cochlear implant, wherein the electrode array portion of the implant is provided with one or more sensors to detect various features of the electrode array during insertion and to provide feedback to the surgeon during implantation. The sensor uses a sensor array to collect intraoperative information on the state of the electrode array during insertion. For example, if configured with an array of strain sensors, flexing of the electrode array can be detected at any point along the length of the electrode array. This allows for reconstruction of the pose of the electrode array during insertion and detection of contact or possible contact with the inner walls of the cochlea.

Disclosed herein as a second aspect of the invention is a system for interpreting the signals received from the sensors and providing intraoperative feedback to the surgeon. The system is capable of determining the deflection, and therefore the overall pose of a cochlear implant electrode array from noisy sensor data of a sensing array having a plurality of strain sensing elements. Additionally, the system has the capability of providing surgical planning capabilities based on a surgical simulator model. In various embodiments, the system may use analytical models, machine learning models or a combination thereof to interpret the raw sensor data and to determine the pose of the electrode array. In addition, the system is capable of making predictions of a positive or negative surgical outcome and possible steps that can be taken by the user to improve the probability of a positive surgical outcome.

Disclosed herein as a third aspect of the invention is a cochlear implant surgery simulator and training system. The system consists of a mock scala tympani to test cochlear implant electrode array insertion, in combination with a sensor system attached to the cochlear implant electrode array to report surgeon performance and allow the surgeon to optimize their technique.

### BRIEF DESCRIPTION OF THE DRAWINGS

By way of example, a specific exemplary embodiment of the disclosed system and method will now be described, with reference to the accompanying drawings, in which:

FIG. 1 is a schematic representation of a middle ear, showing ideal positioning of a cochlear implant, and, in particular, the electrode array portion of the implant.

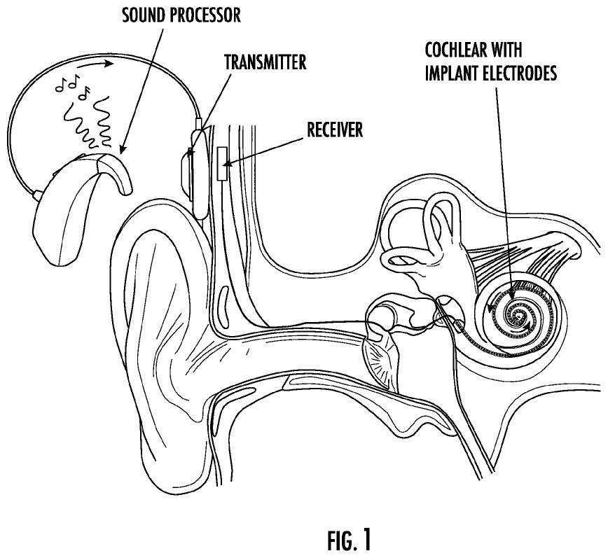

FIG. 2 is a schematic representation of an electrode array having an integrated sensing array.

FIG. 3A is a schematic representation of a strain sensor of the type that could be used in the embodiments disclosed herein, in a neutral position; FIG. 3B is a schematic representation of the strain sensor in an elongated position.

FIG. 4 is a block diagram of the smart sensing system utilized with the cochlear implant/sensing array of FIG. 2.

FIG. 5 is a schematic representation of an electrode array/integrated sensing array showing force and position vectors acting on the electrode array.

FIG. 6 is a block diagram of an analytical model used to estimate force and position vectors from raw data from the sensing elements of the instrumented electrode array.

FIG. 7 a block diagram using a trained machines learning model to estimate force and position vectors from raw data from the sensing elements of the instrumented electrode array.

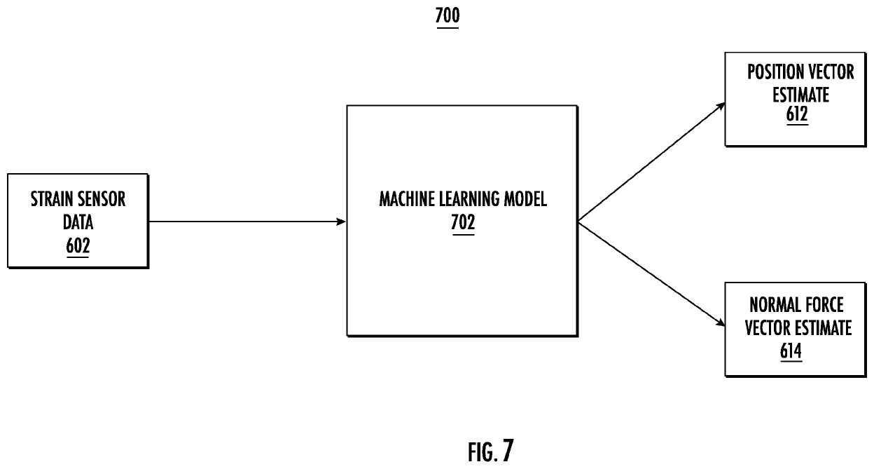

FIG. 8 is a block diagram of a portion of the system for performing dimensionality reduction to produce a high-order state vector representing the pose of the electrode array.

FIG. 9 is an exemplary state tree showing transitions from one state to another or the electrode array, based on the state vectors.

FIG. 10 is an exemplary 3D hollow model of the scala tympani used by the training aspect of the invention for practice electrode insertion.

FIG. 11 is a block diagram of a system used for training and practice electrode insertion.

### DETAILED DESCRIPTION

Disclosed herein as a first aspect of the invention is an instrumented electrode array of a cochlear implant wherein the electrode array is configured with one or more sensing elements formed into a microfabricated thin-film sensing array. The sensing elements are preferably microfabricated as thin-film sensors and integrated with the electrode array. Various types of sensors may be deployed as part of the sensing array, including, for example, strain sensors (i.e., resistive, capacitive, or crack-based), force/pressure sensors (capacitive or electrochemical diaphragm), temperature sensors, proximity sensors, optical sensors, optical spectrometry, reflectometry, imaging, coherence tomography based on integrated optical fibers or waveguides, chemical detection, etc. The sensing array may also integrate microfluidic capabilities to enable sensing or to aid surgery via drug delivery or to relieve fluid pressure in the scala tympani.

In various embodiments, one or more different types of sensing elements may be deployed as part of the thin-film sensing array to provide multiple sensing modalities within a single sensing array. Additionally, like sensing elements may be oriented differently on the thin-film. For example, a plurality of strain sensing elements may be oriented in different directions on the thin-film such as to be capable of detecting elongation or compression along multiple axes.

The invention is described herein the context of a microfabricated interdigitated electrode array used as a strain sensor, however, as would be realized by one of skill in the art, any type of sensor previously mentioned or known in the art is contemplated be within the scope of the invention.

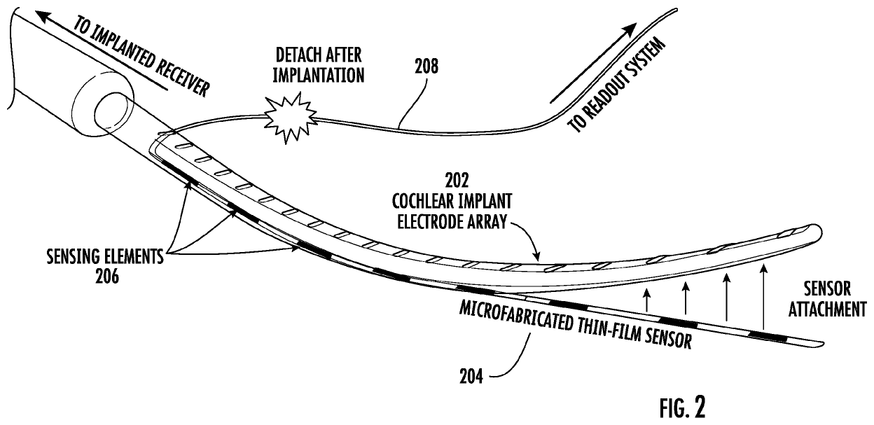

FIG. 2 is a schematic of a specific instantiation of the thin-film microfabricated sensor array 204 designed to attach to or integrate with a cochlear implant electrode array 202 and communicate with a readout system (described below) via flexible cable 208. Microfabricated sensor array 204 comprises a plurality of sensing elements 206 deployed along a length of the array. The actual number of sensors 206 deployed as part of the microfabricated sensor array 204 is dependent on the sensing modality and the desired features to be extracted from the data. For example, in one embodiment wherein the sensing elements 206 are strain sensors, one sensing element 206 may be deployed between each pair of electrodes in electrode array 202 such as to detect flexing of the electrode array 206 along any part of its length.

In one embodiment, the microfabricated thin-film sensor 304 is designed to be disconnected from the readout system after implantation by severing cable 208 as shown in FIG. 2, thereby leaving the inert sensor implanted. In other embodiments, the sensor array 204 may remain active to perform post-operative monitoring or be removed following surgery.

In one embodiment, the microfabricated thin-film sensing array 204 may be attached to a cochlear implant electrode array 202 after manufacturing via an assembly process. For example, sensor array 204 may be joined to the electrode array 202 using a silicone adhesive. In an alternative embodiment, sensing array 204 may be integrated into the manufacturing process of electrode array 202 by including it, for example, in an injection molding process used to produce electrode array 202. The dimensions of the thin-film sensing array 204 may be varied to match the dimensions of various cochlear implant electrode arrays 202 from different manufacturers.

The construction of the thin-film sensing array 204 is not limited to a single material platform. The one embodiment, the sensing elements 206 use platinum traces embedded in a Parylene C insulation to form an interdigitated electrode array strain sensor. These materials are largely equivalent to other common biocompatible materials such as aluminum and gold to form traces and other polymer insulators, for example, Parylenes, Siloxanes, Polyamide, SU-8, etc. Similarly, an optical waveguide may be implemented with a Parylene C core and silicone cladding (e.g., Parylene photonics), but may also be composed of other materials (e.g., SU-8, Ormocers, etc.).

The microfabricated thin-film sensing array 204 may be as previously described and may utilize one or more optical, electrical, electrochemical or microfluidic systems. One exemplary embodiment of the thin-film sensing array 204 is a metal strain gauge based on an interdigitated electrode array capacitive strain sensor. A second exemplary embodiment of the sensing array 204 is an integrated photonic waveguide to perform fiber optical coherence tomography intraoperatively.

In one embodiment, one or more interdigitated electrode array (IDE) capacitive strain sensors may be utilized as sensing elements 206 on the thin-film sensing array 204. The sensing elements 206 and the overall thin-film sensing array 204 may be fabricated as described in Provisional Patent Application No. 63/324,839, to which this application claims priority. The contents of this application are incorporated herein in their entirety.

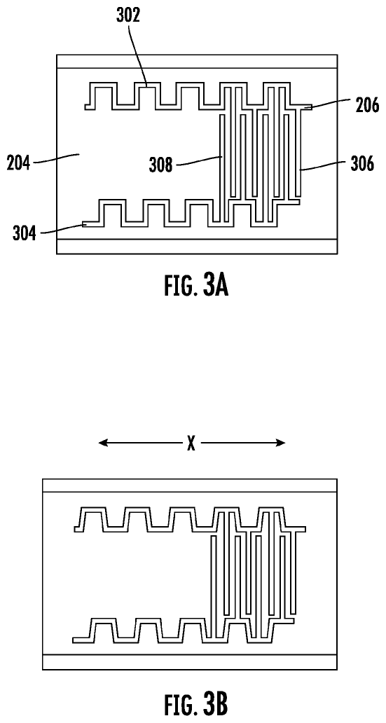

FIGS. 3A,3B are schematic representations of an exemplary strain sensor of the type which may be used as sensing element 206. FIG. 3A shows sensing element 206 in a neutral position, while FIG. 3B is a schematic representation of sensing element 206 in an elongated position. Sensing element 206 comprises a first trace 302 electrically-coupled to a first sub-plurality of the fingers 306 and a second trace 304 electrically-coupled to a second sub-plurality of the fingers 308, wherein the first and second sub-pluralities are exclusive of each other. In embodiments disclosed herein one trace is a ground trace and the other trace is a sense trace. Preferably, fingers in the first sub-plurality will be disposed between two fingers in the second plurality (except at the ends of the array) and vise-versa, thus forming a set of interdigitated fingers. Each of fingers 306, 308 may comprise a stack consisting of a layer of polymer, for example, Parylene C and a thin-film electrically-conductive material, for example, platinum or gold. The polymer layer supporting each finger allows the elongation of the overall device along longitudinal axis X, while still allowing the device to be fabricated using high-volume MEMS fabrication techniques. Fingers 306, 308 may be encapsulated in a protective layer comprised of, for example, PDMS. Traces 302, 304 make use of in-plane trace routing to reduce the stiffness of the sensor along the longitudinal axis of elongation (“X”).

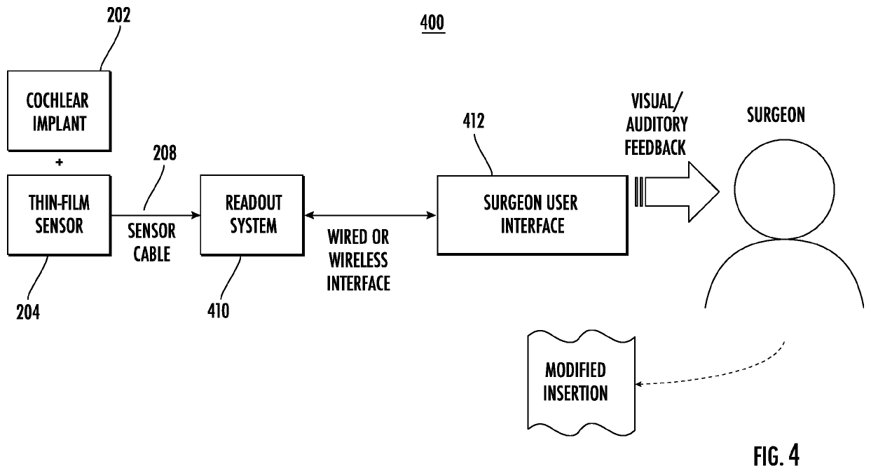

FIG. 4 is a block diagram of a smart sensor system 400 disclosed as a second aspect of the invention. Smart sensor system 400 is composed of multiple components: an electrode array of a cochlear implant 202, a microfabricated thin-film sensing array 204, a readout system 410 which digitizes and processes the signals received from thin-film sensing array 204 via sensor cable 208, and a surgeon (user) interface 412. In various embodiments, system 400 may be stand-alone or integrated into a larger surgical system, for example, a robotically-assisted surgical system. Various systems are also known wherein intraoperative feedback may be provided by the electrodes in the electrode array. In various embodiments, the microfabricated thin-film sensing array 204 described herein and integrated with electrode array 202 may be used in conjunction with or independently of any sensed information collected from the electrodes in electrode array 202.

The readout system 410 is composed of several discrete components, preferably integrated on a printed circuit board. Readout system 410 may include any required input/output interfaces, an amplifier and digitizer circuits that may be required to operate the thin-film sensor array 204, including, but not limited to: resistive, capacitive, or impedance measurement circuits, voltage or current sources for electrical sensors, or laser diodes, spectrometers, optical filters, and power meters for optical systems. The readout system 410 also contains a microcontroller to process and store the data, as well as power control (voltage regulators or battery circuitry) and wired or wireless communication circuitry.

The user (surgeon) interface 412 provides feedback to the surgeon and displays the information acquired by the readout system 310 to the surgeon. The feedback and display may consist of audible cues and/or a visual display of metrics (e.g., wrapping factor or tip force), or a more complex visualization (e.g., a visualization of a 3D pose of the cochlear implant electrode array 202, or the strain or force distribution along the array). User interface 312 may consist of a device with a screen or speakers, or an augmented-reality display.

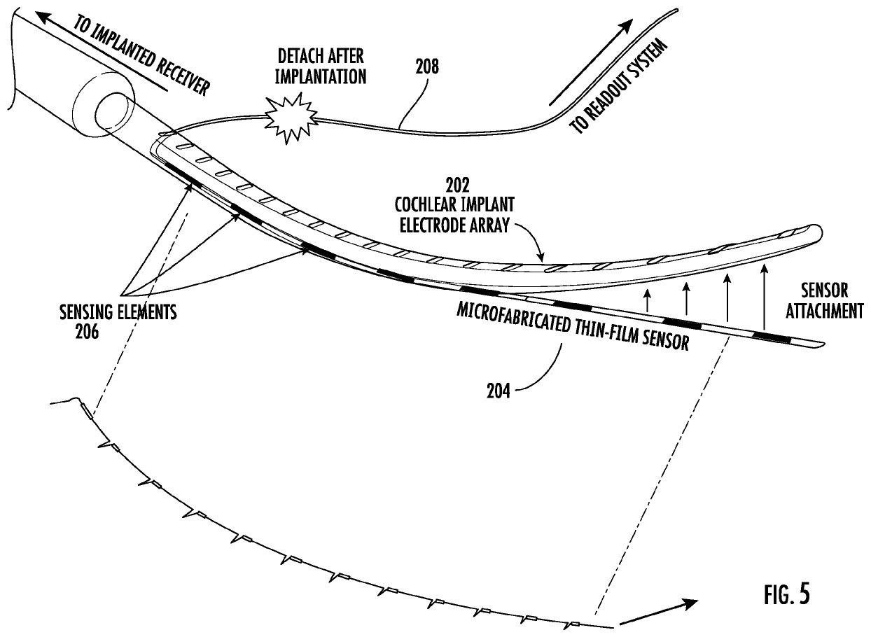

A software system for interpreting the sensor data is disclosed as a second aspect of the invention. In various embodiments, readout system 410 may be configured to derive force and position information from data from the plurality of sensing elements 206. FIG. 5 is a schematic diagram showing the force and position vectors acting on the cochlear implant electrode array based on the output of the plurality of strain sensing elements 206 in sensing array 204. Details of readout system 410 will be discussed in the context of sensing array 204 comprising a plurality of strain sensing elements 206, which may be oriented in different directions on the thin-film. As would be realized, any other type of sensor previously discussed or know in the art could also be used and may result in different types of features being extracted from the data.

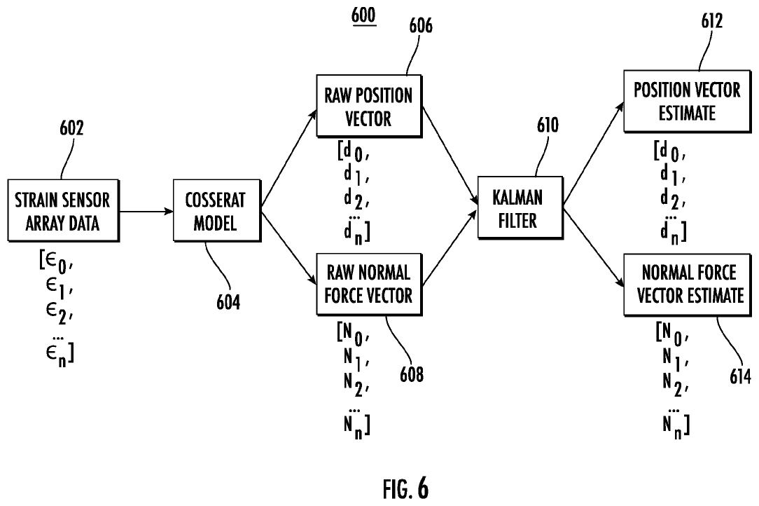

In one embodiment, an analytical model may be used to derive force and position vectors based on the data from sensing elements 206. FIG. 6 shows an exemplary embodiment of an analytical model 600 that could be used for this purpose. Raw data 602 from sensing elements 206 is input to model 600. Given the known mechanics of the cochlear implant electrode array 202 (i.e., known geometry, bending stiffness, shearing stiffness, and second moment of inertia), the Cosserat rod model 604 can be used to calculate the position and orientation along the length of the electrode array 202, as well as the external forces acting on the electrode array 202 to induce that conformation from the strain along the rod length. Estimates of these properties are calculated from strain readings derived from a sensing element of a sensing array disposed along the length of the cochlear implant electrode array 202 to produce a raw position vector 606 and raw force vector 608. A Kalman filter 610 may be used to estimate the underlying state of the system given sensor noise, resulting in estimated position vector 612 and estimated force vector 614.

In an alternate embodiment, a machine learning-based model 700 may be used to determine the position vector estimate 612 and the normal force vector estimate 614. Machine learning-based model 700 may use machine learning model 702 which is trained on ground truth position and force information derived from simulated cochlear implant surgeries or training sessions performed on cochlear models. Machine learning model 702 may be of any known architecture.

The position and normal force vectors produced by analytic model 600 or machine learning-based model 700 may be used to create an anatomically accurate pose estimation of electrode array 202 as it is inserted into the cochlea and this pose estimation can be visualized to the surgeon via user interface 412. In addition, alarms may be raised to the user when the normal force vector indicates that certain forces have exceeded predetermined thresholds or when the position vector indicates a position deviation of electrode array 202 and may also be delivered via user interface 412.

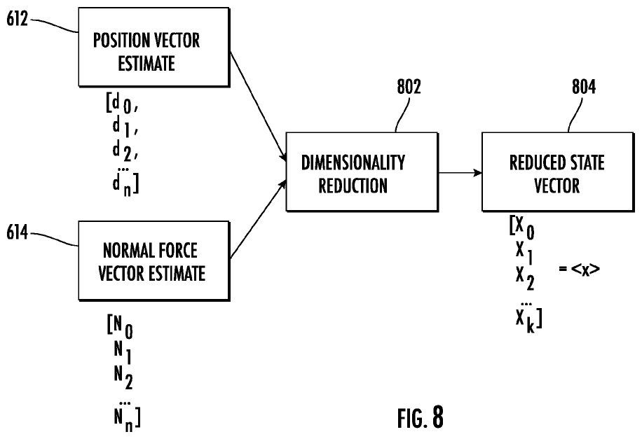

FIG. 8 is a block diagram of the second part of readout system 310 in which dimensionality reduction techniques are used to generate a lower-dimensional, higher-order state vector representation 804 of electrode array 202, based on the estimated position vector 612 and the estimated normal force vector 614. In preferred embodiments, the dimensionality reduction will result in one or more higher-order features which are indicative of a positive clinical outcome. In one embodiment, the dimensionality reduction 802 is accomplished using principal component analysis (e.g., 5 component PCA). In other embodiments, dimensionality reduction 802 may be accomplished by use of an autoencoder or a machine learning model trained by extracting ground truth features from the estimated position vector 612 and estimated normal force vector 614 for implantation surgeries which have resulted in a positive clinical outcome.

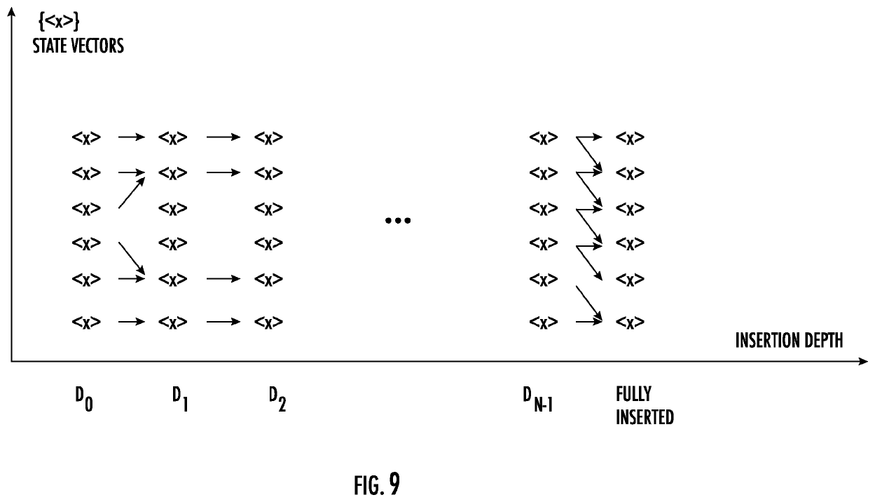

Readout system 310 may also include a surgical route planning component wherein electrode states are discretized by insertion depth. A state tree is shown in FIG. 9. Transitions between electrode states indicate discretized surgeon actions (i.e., 100 micron insertion at a specific insertion angle: [0, 10, 20, 30, etc. degrees]). Fully inserted states are rated in quality via a wrapping factor or other placement metrics (placement quality indicated by color coding with red=poor, orange=fair, green=good). Prior states are ranked by the highest quality state that can be reached from the current state. Intermediate states are also rated via additional metrics (i.e., transitory insertion force). Using a search algorithm, the state tree shown in FIG. 9 can be used for surgical path planning to identify an optimal series of surgical actions to achieve a positive clinical outcome and avoiding intermediate states associated with a high probability of surgical trauma. Furthermore, if the surgeon deviates from the optimal path, a new search from the current state vector can predict an optimal recovery route.

For surgical planning, insertion of the electrode array 202 in a 3D model of a cochlea can be simulated using the Simulation Open Framework Architecture (SOFA). This simulation approach can be tailored to individual patients using 3D geometry of the scala tympani reconstructed from CT scans. In one embodiment, the insertion is discretized by insertion depth in intervals of 100 micrometers, although other discretization intervals may be used, which are chosen based on the available computation resources. At each step, a series of possible actions may be simulated to form a branching decision tree of surgical states. These decisions are simulated in discretized steps: insertion speed (i.e., [0.5, 1, 2 mm/s]), and insertion angle (i.e., Δθ=[−15, −10, −5, 0, 5, 10, 15 degrees]). Finer discretization intervals may be chosen if computational resources are available.

End states of the decision tree of surgical states (full insertion) are evaluated by placement metrics such as wrapping factor. Intermediate states are evaluated by the optimal placement that can be reached from each state. Intermediate state rankings are penalized by the peak insertion force at that state to avoid insertion trajectories that result in large transient forces which may result in surgical trauma.

In one embodiment, efficient surgical planning can be achieved via a search algorithm to identify a path to an optimal end state while avoiding intermediate states causing high insertion force. Given an arbitrary initial state (i.e., if surgical trajectory has deviated from the optimal path), a new search can be performed in the decision tree to identify the optimal recovery path. At each step of insertion, a minimally traumatic optimal placement path is defined in prescriptive steps of Δθ insertion angle change and insertion speed. In various alternative embodiments, the surgical route planning component may be implemented using a trained machine learning model where in the ground truth is indicated by surgical actions which resulted in positive clinical outcomes. The machine learning model may recommend an optimal surgical path and may also recommend remedial or next actions to be taken in the event that the position or force vectors 612, 614, or the higher-order features 804 previously discussed indicate pending trauma.

A cochlear implant training system is disclosed as a third aspect of the invention. The training system uses a cochlear implant having an instrumented electrode array as previously described and as shown in FIG. 2. The training system serves a two-fold purpose. First, the training system provides training data for the training of the multiple machine learning models previously discussed herein. Second, the training system provides feedback regarding the performance of surgeons during the electrode implantation procedure, thereby enabling the surgeon to refine and optimize their insertion technique.

In one embodiment, the training system utilizes a hollow model of the scala tympani of the cochlea. The model may be created via a 3D-printing process and, in some embodiments, may be transparent such as to allow observation of the pose of the electrode array at various points during insertion process. During the training process, the model may be masked from the surgeon to better simulate insertion of the electrode rating an actual cochlea which, of course, is done “blind”.

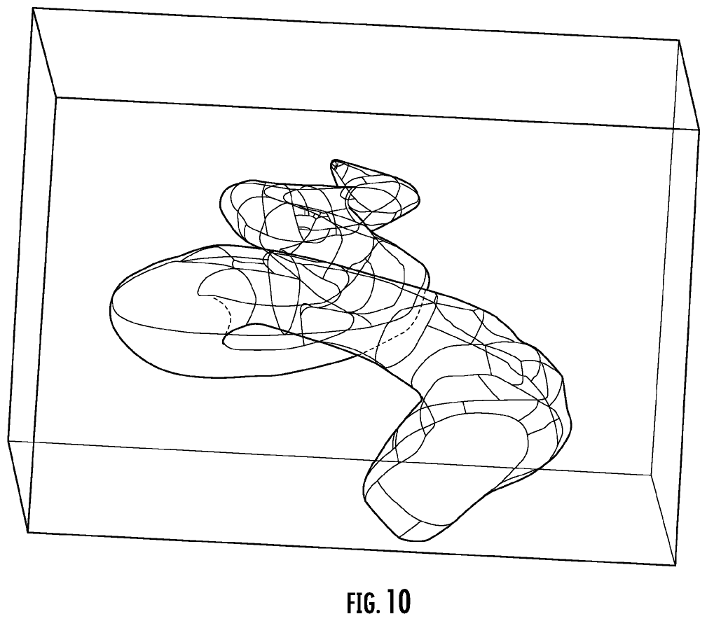

An exemplary 3D-printed mold of a scala tympani is shown in FIG. 10. A 3D-printed scala tympani can be built using stereolithographic 3D printing. The 3D models can be designed to be anatomically accurate based on scans of actual human scala tympani, such as those available in the open-source OpenEar database. 3D models may also be created using imaging data collected from patients on whom the surgeon is to operate, such that the surgeon can practice on a model of the patient's cochlea prior to the actual procedure.

Training data may be created for the multiple machine learning models previously discussed by observing the ground truth position of the electrode array during various points of the insertion process and associating those positions with features, force and position vectors and the reduced dimension, higher-order state vector extracted from the data received from the multiple sensing elements in the instrumented electrode array. Data may also be collected regarding the change in state from one insertion point to the next insertion point and the force a position vectors associated with each state.

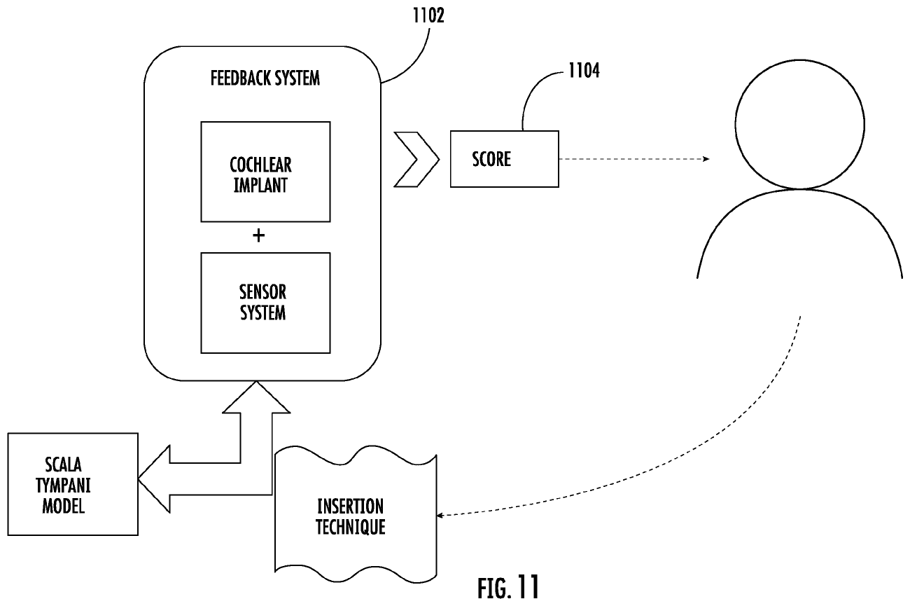

The 3D-printed scala tympani is paired with a feedback system 1102, shown in block diagram form in FIG. 11, which is capable of recording the insertion and reporting quantitative metrics in the form of a score 1104 indicating the quality of the insertion. In one embodiment, two specific metrics comprising the score 1104 may include a peak insertion force and wrapping factor. The wrapping factor provides a metric of how tightly or loosely wrapped an electrode array is relative to the modiolus. A high wrapping factor closer to 100% means that the array is close to the lateral wall, while a smaller number indicates the array is wrapped more tightly relative to the modiolus. System 1102 reads data from sensing elements 206 in sensing array 204 and reports the metrics during practice implantation procedures. As would be realized by one of skill in the art, other metrics may be reported in addition to or in place of the peak insertion force and the wrapping factor.

In various embodiments, the practicing surgeon may have access to data generated by the software system previously described, for interpreting and displaying the data received by sensing elements 206 during the practice implantation procedures. In other embodiments, the surgeon may perform the procedure without the aid of the interpretation of the data provided by instrumented electrode array, which is potentially useful when generating the training data for the machine learning models.

The invention is contemplated to include both the instrumented cochlear implant and the system for analyzing the data and providing intraoperative feedback to the surgeon, including recommending actions to raise the probability of a positive clinical outcome. As would be realized by one of skill in the art, many variations on the system and the device disclosed herein are possible and are contemplated to be within the scope of the invention, which is defined by the claims which follow.

1. A system comprising:
a 3D model of the scala tympani of a cochlea;
a processor; and
software that, when executed by the processor, causes the system to:
receive data from one or more sensing elements of an instrumented electrode array of a cochlear implant during insertion of the electrode array in the 3D model;
estimate a normal force vector comprising forces acting along a length of the electrode array;
estimate a position vector comprising a position of one or more segments of the electrode array; and
provide a feedback score comprising one or more metrics derived from the estimated normal force and position vectors.

2. The system of claim 1 wherein the 3D model is transparent to allow observation of an actual position and state of the electrode array at discrete points during the insertion of the electrode array into the 3D model.

3. The system of claim 2 wherein the observed position and state of the electrode array and the data received from the one or more sensing elements are used as training data for a first machine learning model used to output the normal force and position vectors.

4. The system of claim 2 wherein the normal force and position vectors are used as training data for a second machine learning model used to perform dimensionality reduction of the normal force and position vectors to produce a lower-dimensional, higher-order state vector representation of the electrode array.

5. The system of claim 4 wherein the higher-order state vector represents features of the force and position vectors indicative of a high probability of a positive clinical outcome.

6. The system of claim 4 wherein the higher-order state vector representation to is used as training data for a third machine learning model to produce an action space indicative of surgical actions that increase a probability of a positive clinical outcome.

7. The system of claim 1 wherein the metrics comprising the feedback score include peak insertion force and wrapping factor.

8. The system of claim 1 wherein a surgeon performs a practice insertion of the electrode array without observing the actual position of the electrode array via the transparent model.

9. The system of claim 8 wherein the surgeon receives no feedback from the system during practice insertion of the electrode array.

10. The system of claim 1 wherein the one or more sensing elements of the electrode array comprise strain sensors.

11. The system of claim 1 wherein the software further causes the system to:
provide feedback to a user when the force vector indicates that one or more portions of the electrode array exhibit forces that exceed predetermined thresholds.

12. The system of claim 1 wherein the software further causes the system to:
provide feedback to a user when the position vector indicates that one or more segments of the electrode array exhibit a position deviation.

13. The system of claim 1 wherein the software performs the further function of:
providing a visualization of the position and state of the electrode array as it is inserted into the model.

14. The system of claim 1 wherein the 3D model is created using a 3D printing process.

15. The system of claim 1 wherein the model is created based on imaging data from an actual recipient of the implant.
